(19) United States
(12) Patent Application Publication
(10) Pub. No.: US 2026/0013741 A1
(43) Pub. Date: Jan. 15, 2026

(54) SYSTEMS, DEVICES, AND METHODS FOR MONITORING PHYSIOLOGIC CONDITIONS DURING A MEDICAL PROCEDURE

(71) Applicant: Certus Critical Care, Inc., Salt Lake City, UT (US)

(72) Inventors: Myra FABRO, San Francisco, CA (US); Mariel FABRO, San Jose, CA (US); Ruth BEEBY, Santa Clara, CA (US)

(21) Appl. No.: 18/657,242

(22) Filed: May 7, 2024

Related U.S. Application Data
(63) Continuation of application No. PCT/US2022/049335, filed on Nov. 8, 2022.
(60) Provisional application No. 63/277,428, filed on Nov. 9, 2021.

(51) Int. Cl. A61B 5/0215; A61B 5/00

(52) U.S. Cl. CPC A61B 5/0215; A61B 5/4064; A61B 5/4255; A61B 5/6852

(57) ABSTRACT

A device to monitor a physiologic condition of a patient during a medical procedure is described herein. The device may comprise a sleeve, a sensor housing, and an elongate body. The sleeve may comprise a lumen therethrough and a window. The sensor housing may comprise a sensor therein. The sensor housing may be coupled to the sleeve such that the sensor is aligned with the window. The elongate body may be positioned within the lumen of the sleeve. The sensor may be aligned with an outer surface of the elongate body.

### CROSS-REFERENCE TO RELATED APPLICATIONS

This application is a continuation of International Application No. PCT/US2022/049335, filed on Nov. 8, 2022, which claims priority to U.S. Provisional Application No. 63/277,428, filed on Nov. 9, 2021, each of which is hereby incorporated by reference in its entirety.

### GOVERNMENT SUPPORT

This invention was made with government support under grant FA8650-20-2-6116 and W81XWH-21-C-0058 awarded by the United States Air Force/Air Force Material Command. The government has certain rights in the invention.

### TECHNICAL FIELD

This invention relates generally to the field of integrating one or more sensors into a catheter for monitoring physiologic conditions during a medical procedure.

### BACKGROUND

Catheters serve a wide range of functions and may be used in various procedures. For example, catheters may be used in procedures such as cardiovascular procedures, urological procedure, gastrointestinal procedures, neurovascular procedures, ophthalmic procedures, etc. Many of these medical procedures may be assisted by one or more sensors that provide information to a user (e.g., surgeon, operator, etc.) at the point of intervention.

Conventional catheters have several drawbacks. Many existing catheters are stand-alone catheters. Such catheters may need separate sensing devices that are used in conjunction with the stand-alone catheters to provide information to a user during a procedure. This may make the system used for performing the medical procedure bulky. Furthermore, additional personnel may be required (e.g., to deploy the separate sensing devices and monitor them) to perform the medical procedure. Some recently developed catheters have sensors incorporated into them, but these catheters suffer from several drawbacks, such as decreased accuracy and limited availability with respect to sensor-type. For example, conventional catheters incorporating sensors do not utilize solid-state sensors (e.g., solid-state pressure sensors, etc.) for absolute pressure measurements. Instead, conventional catheters incorporating sensors typically use fluid columns for pressure measurement. However, fluid columns suffer from several drawbacks such as, for example, excessive noise, over and or under dampening of the signal, high susceptibility to noise during any movement of the patient or the arterial line, clots and or blockages in the arterial line, lack of portability, etc.

Moreover, conventional catheters are typically subjected to various forces (e.g., bending forces) and contact with objects within a patient's body during insertion and use. Conventional catheters have been unable to prevent these forces from affecting the precision of the sensor measurements and/or from preventing unwanted contact between the sensors and objects within a patient's body that may also result in inaccurate measurements.

Therefore, additional devices with integrated sensors to monitor physiologic conditions while simultaneously providing accurate and precise sensor measurements are needed.

### SUMMARY

Systems, devices, and methods for monitoring physiologic conditions during a medical procedure are described herein. In some variations, a device configured to monitor a physiologic condition of a patient during a medical procedure may comprise a sleeve, a sensor housing comprising a sensor therein, and an elongate body. The sleeve may comprise a lumen therethrough and a window. The sensor housing may be coupled to the sleeve such that the sensor is aligned with the window. The elongate body may be positioned within the lumen of the sleeve. The sensor may be aligned with an outer surface of the sensor body.

In some variations, the elongate body may comprise an opening and the sensor housing may be positioned within the opening of the elongate body. In some variations, the sleeve may be configured to add stiffness to the elongate body local to the sensor. In some variations, the sleeve may include a feature configured to distribute bending forces acting on the sensor. The sleeve may comprise a slit. The slit may have a spiral geometry.

In some variations, the sensor housing may comprise a polymer. In some variations, the sensor may be encapsulated in the sensor housing. In some variations, the sleeve may be configured to seal the sensor to the elongate body. In some variations, the sensor housing may comprise a strut coupled to the sleeve. In some variations, the strut may be coupled to the sleeve in the window. The sensor housing may comprise a sealed body portion containing the sensor.

In some variations, the length of the strut may be between 0.005 inches and about 0.02 inches. In some variations, the sensor housing may be tubular in shape. In some variations, the sensor housing may further comprise a cantilever mount to mount the sensor on the sensor housing and a cantilever arm to support the cantilever mount. The cantilever mount may be aligned with the window. The cantilever mount may be positioned at a depth of about 0.02 inches and about 0.06 inches from an outer surface of the sleeve. In some variations, the elongate body may comprise an inner layer and an outer layer. A sensor wire associated with the sensor may be routed between the inner layer and the outer layer.

In some variations, the device may be a blood flow control device and the physiologic condition may be blood pressure. The elongate body may further comprise an expandable member coupled to the elongate body. In some variations, the device may be configured to be advanced into one or more blood vessels in the brain. The device may be configured to identify transition of the elongate body from a first part of the brain to a second part of the brain. In some variations, the device may be configured to monitor a functioning of a gastrointestinal tract. In some variations, the elongate body is an endotracheal tube.

### BRIEF DESCRIPTION OF THE DRAWINGS

FIG. 1A illustrates an exemplary variation of a sensor integrated into an elongate body.

FIG. 1B depicts an exemplary variation of a sensor sleeve and a sensor housing.

FIG. 1C depicts the exemplary sensor housing of FIG. 1B with an opening that allows the housing to be entirely or partially filled with a polymer.

FIG. 2A illustrates another exemplary variation of a sensor integrated into an elongate body.

FIG. 2B depicts a cross-sectional view of the exemplary variation shown in FIG. 2A.

FIG. 3 illustrates an exemplary variation of a cross-section of a portion of an elongate body.

FIG. 4 illustrates an exemplary variation of a blood flow control device including an elongate body with integrated sensors.

FIG. 5 illustrates an exemplary variation of a blood flow control system including an elongate body with integrated sensors.

### DETAILED DESCRIPTION

Non-limiting examples of various aspects and variations of the invention are described herein and illustrated in the accompanying drawings.

Systems and devices for monitoring physiologic states in patients and physiologic conditions during a medical procedure are described herein. More specifically, devices comprising an elongate body (e.g., a catheter) with one or more integrated sensors for monitoring physiologic conditions during a medical procedure and/or or monitoring patients physiology over prolonged periods of time during routine and critical medical care are described herein.

Measuring physiologic conditions in real-time at a point of intervention (e.g., during a medical procedure) may provide valuable data that may guide treatment decisions. For example, changes to physiologic conditions during a medical procedure may provide information about the patient, such as for example, whether the patient needs medication, the amount of medication that may be needed, whether the patient needs intravenous (IV) fluids, etc. In cases in which a balloon catheter is used during a medical procedure, measuring the physiologic conditions in real-time may provide information that may be used to make changes to the balloon (e.g., amount of expansion and/or contraction of the balloon, etc.) in order to, for example, impact a patient's blood flow and/or blood pressure.

Medical practitioners (e.g., surgeons, operators, users, etc.) use existing catheters in conjunction with sensor devices to monitor physiologic conditions in real-time. Some recently developed catheters include sensors that measure physiologic conditions, but these catheters typically utilize traditional sensors, not solid-state sensors. For example, some existing catheters include pressure sensors that utilize fluid columns to measure the pressure. Typically, these pressure sensors may be positioned outside the body of the patient within a device that includes the catheters or as a stand-alone pressure sensor/device that is connected to the catheters. The pressure in the body is transduced through the fluid column within the catheter to the pressure sensor. In these cases, medical devices may need to be modified to accommodate the pressure sensing columns. Moreover, fluid columns can be cumbersome to use and may result in inaccurate sensing data. For example, fluid columns are known to clot with blood. Additionally, as the length of the fluid column increases, the sensor signals may begin to dampen, which may result in inaccuracies and inability to measure the true maximum values and minimum values of a cyclical or changing physiologic condition. Additionally, movements of the fluid column and/or inadvertent contact with the fluid column may lead to excessive noise that can result in inaccurate pressure readings. Thus, use of fluid columns can be particularly challenging in circumstances where portability and/or field use are desirable.

In addition to the above, existing catheters that include pressure sensors that utilize fluid columns take time to set up. For instance, during set up, a user (e.g., surgeon, operator, etc.) may have to flush the fluid columns with saline to ensure that there is no air in the fluid column. The user may also have to continuously and/or intermittently continue to flush the fluid columns to ensure that clots are not building up in the fluid columns. This can make such catheters cumbersome to use. The use of fluid columns may be particularly challenging in circumstances where blood pressure measurement and/or pressure monitoring may be needed quickly without significant set up time. Furthermore, fluid columns may run along the surface of the catheter or inside the catheter. All of this may make the devices bulky and challenging to use.

Additionally, during use in the body, catheters may be subjected to various forces, such as, for example, bending forces, that may negatively impact an integrated sensor's ability to obtain accurate and precise measurements. Moreover, in some instances, sensors may be subject to contact with foreign materials in a patient's body, which may damage the sensors and/or decrease the sensor's accuracy. Accordingly, medical devices including catheters with integrated sensors (e.g., solid-state sensors) that are shielded from unwanted forces and unwanted contact with objects within the body, yet still provide the benefits of integrated sensing capabilities with accurate measurements are needed.

Devices and systems described herein may include medical devices with integrated sensors that provide accurate readings of a patient's physiology despite exposure to external forces. Devices and systems described herein may include medical devices with solid-state sensors (e.g., solid-state pressure sensors) that provide absolute pressure measurements. The devices described herein may comprise an elongate body and one or more sensors. The one or more sensors may be contained in a sensor housing and may be coupled to, or integrated with, an elongate body using a sensor sleeve. The sensor housing and/or a sensing surface of a sensor may be aligned with an external surface of the elongate body and may be coupled to the elongate body in a manner that distributes applied forces and reduces exposure to external objects to minimize and/or prevent these forces and objects from negatively impacting sensor accuracy. The sensor housing and/or sleeve may be sealed to the elongate body, thereby protecting the sensors from foreign materials that may additionally negatively impact sensor accuracy. At least a portion of a top surface of the sensor and/or sensor housing may be exposed through an opening and/or a window so as to allow the sensors to measure physiologic conditions. In variations where the surface of the sensor includes a polymer layer, or when the sensor is encapsulated within a polymer, exposure of the sensor to physiologic conditions may occur through the polymer layer or polymer encapsulation. Additionally, in some instances, the sensors may be integrated into the elongate body such that the sensors and/or any associated components utilize the space in the elongate body efficiently, thereby contributing to a reduced form factor of the elongate body and thus the device.

### Sensor Integration

### Elongate Body

The devices described herein may comprise an elongate body comprising one or more sensors (e.g., one, two, three, four, five, or more), and in particular, the sensors may be integrated into the elongate body. The elongate body may comprise a shaft sized and shaped for placement within a body of a patient (e.g., at a point of intervention such as in a blood vessel, parenchyma of the brain, esophagus, stomach, small intestine, etc.). In some variations, the elongate body may be steerable. For example, in some variations, the elongate body may be mechanically coupled to knobs, levers, pullwires, and/or the like that may be used to steer or otherwise deflect a distal end of the shaft of the elongate body. In some variations, the elongate body may include one or more lumens therethrough. The lumen(s) may be partial lumen(s) (e.g., open on one end) and may be disposed within or lie within the movable shaft. The one or more lumens (e.g., two, three, four, or more) may serve any desired purpose. For example, in some variations, the lumens may be used for transmitting fluids to and from a patient's body and/or other components coupled to the elongate body, advancing and/or steering a guidewire into a desired location, housing other components (e.g., sensor wires, pressure sensing columns, imaging devices such as endoscopes, etc.) etc. In some variations, the lumen(s) may include an intake lumen and an exhaust lumen to deliver fluid and/or compressed gas through the elongate body.

As mentioned above, the elongate body may be sized and shaped for advancement to, and placement at least partially within, a target location of the patient's body. The elongate body may have any diameter and length suitable for advancement to the target location. For example, the elongate body may have a diameter between about 2 mm and about 36 mm. In some variations, the diameter may be for example between about 3 mm and about 25 mm, between about 4 mm and about 20 mm, or between about 5 mm and about 15 mm (including all values and sub-ranges therein). In some variations, the diameter may be for example between about 6 mm and about 10 mm. The elongate body may have a length between about 1 cm and about 110 cm. In some variations, the length may be for example between about 10 cm and about 105 cm, between about 20 cm and about 100 cm, between about 30 cm and about 90 cm, between about 40 cm and about 80 cm, or between about 50 cm and 70 cm (including all values and sub-ranges therein).

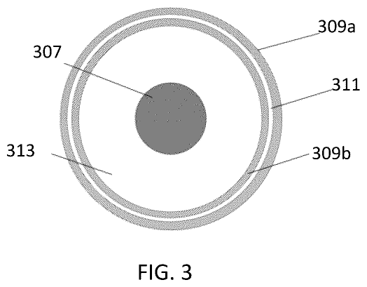

In some variations, the elongate body may comprise multiple layers. For example, one or more portions of the elongate body may comprise a plurality of layers (e.g., two, three, four, or more), and all portions of the elongate body may comprise the same layers, or the layers may differ between different portions of the elongate body. FIG. 3 illustrates an exemplary variation of a cross-section of a portion 309a of the elongate body 302. The elongate body 302 may include an inner layer 309b and an outer layer 309a. The inner layer 309b may be an inner shaft and the outer layer 309a may be an outer shaft such that the diameter of the outer shaft is greater than the diameter of the inner shaft. The elongate body may include an annular space 311 between the outer layer 309a and the inner layer 309b, and may comprise a lumen 313. The elongate body may also include a core 307, which may, for example, comprise a metal alloy, and may be positioned within the lumen 313. In other variations, the elongate body may comprise a single layer that may include one or more lumens therethrough. The elongate body, and/or any layer of the elongate body, may be formed from any suitable biocompatible material, such as, for example, Polytetrafluoroethylene (PTFE), polyimide, and Pebax®, a combination thereof, and the like.

As discussed above, one or more sensors may be integrated into the elongate body. One or more sensor wires associated with the sensor(s) may be routed through the elongate body. For example, in some variations, one or more sensor wires may be positioned within or otherwise routed through the annular space 311. This may advantageously decrease the size and/or complexity of the elongate body by allowing for the elongate body to have a single lumen (i.e., within the inner layer 309b) (instead of a multi-lumen extrusion). Accordingly, this may contribute to reduced form factor of the elongate body. In other words, the elongate body may be designed to be compact without the need for extra lumens to route sensor wires. Additionally, routing the sensor wires through the annular space 311 may make the elongate body more kink-resistant. In some variations, sensor wires may be coupled alongside the core 307 and may be protected by a polymeric layer such as Polyethylene terephthalate (PET) for the entire span of the elongate body. In some variations, the sensor wires may be disposed alongside the core 307 (e.g., the sensor wires may run parallel to the core 307). Additionally or alternatively, the sensor wires may surround and/or may be wrapped around the core 307. For example, the sensor wires may surround the core 307 by forming a spiral or helical shape.

The elongate body may comprise an opening and/or a window to receive the sensor(s) via a sensor housing. More specifically, an outer surface or outer layer of the elongate body may comprise the opening and/or the window to receive the sensor housing and thus the sensor. In some variations, the opening and/or window may be a cavity formed within elongate body (e.g., outer layer) that may receive the sensor housing, while in other variations, the opening and/or window may be a through-wall hole formed in the elongate body (e.g., outer layer) and all or a portion of the sensor housing may be positioned within or through the hole. Accordingly, the window and/or opening may receive the sensor via the sensor housing as an inset. In other variations, the elongate body may not comprise an opening and/or window to receive the sensor housing and/or the sensor, and instead, the sensor housing and/or sensor may be positioned on or otherwise contact the outer surface of the elongate body.

The window and/or opening may be sized and shaped to receive the sensor housing. For example, in some variations, the window and/or opening may have a shape corresponding to the shape of the sensor housing and/or the sensor such that the sensor housing and/or the sensor easily fits within the window and/or opening. The sensor housing and/or the sensor may be inserted in the opening and/or window in such a way that the sensor housing and/or the sensor may be aligned with the outer surface of the elongate body. The window and/or opening may further provide a pathway for the sensor wires to access a lumen or annular space within the elongate body for transmission of the sensor signals.

In variations comprising a plurality of sensors, the sensors may be positioned along and around the elongate body in any suitable manner. For example, two or more sensors may be longitudinally aligned along the elongate body. For example, a first sensor and a second sensor may be positioned along the same longitudinal line, but the first sensor may be positioned closer to a proximal end of the elongate body while the second sensor may be positioned closer to the distal end of the elongate body than the first sensor. In some variations, one or more sensors may not be aligned longitudinally and may instead be circumferentially offset from one or more additional sensors such that the sensors are located around the elongate body. In some variations, the circumferential offset (e.g., the angle formed between the longitudinal axes of the sensors) may be between 15 degrees and 345 degrees, such as, for example, 90 degrees, 180 degrees, or 270 degrees. In some variations, one or more sensors may be circumferentially offset from one or more additional sensors but may be aligned between the proximal and distal ends of the elongate body, while in other variations one or more sensors may be circumferentially offset from one or more additional sensors and may be positioned at different locations between the proximal and distal ends of the elongate body.

### Sensor Housing and Sensor Sleeve

The sensors described herein may be contained in, positioned within, and/or encapsulated in a sensor housing. The sensor housing may be coupled to or otherwise affixed to a sleeve. In some variations, the sleeve itself may comprise the sensor housing. The sleeve may be positioned around or may otherwise encircle the elongate body, thereby coupling the sensor(s) to the elongate body.

### Sleeve

The sleeve may be configured to receive the elongate body thereby coupling or integrating the sensor housing, and thus the sensor, to or with the elongate body. The sleeve may include a lumen configured to receive the elongate body, an opening (e.g., a window) such that the sensor housing may be exposed therethrough, and in some variations, a feature to facilitate progressive transition of stiffness between the elongate body and the sleeve.

In some variations, the sleeve may be sized and shaped such that the elongate body may be received within the sleeve. For example, the sleeve may comprise a lumen therethrough with a cross-sectional shape corresponding to a cross-sectional shape of the elongate body and with a diameter larger than an outer diameter of the elongate body. For example, in some variations, the sleeve may be tubular. In some variations, the sleeve may add stiffness to the elongate body around the sensor contained within the sleeve, thereby protecting the sensor from damage and isolating the sensor from sensitivity related errors that may occur due to flexing of the elongate body.

The sleeve may be made from any suitable biocompatible material. For example, the sleeve may be formed from a polymer, such as, for example, Polytetrafluoroethylene (PTFE), polyimide, Pebax®, thermoplastic polymers, a combination thereof, and the like. Exemplary thermoplastic polymers may be a polycarbonate, a polycarbonate/acrylonitrile-butadiene-styrene terpolymer blend, and the like. In some variations, the sleeve may be formed from a metal, e.g., stainless steel, a bondable metal alloy, or a combination thereof. In one variation, the sleeve comprises stainless steel.

In some variations, the sleeve may include a feature that may enable progressive transition of stiffness of the elongate body and may distribute unwanted external forces (e.g., bending forces) acting on the elongate body. For example, in some variations, the sleeve may be stiffer than the elongate body and/or the combination of the elongate body and the sleeve together may make the portion of the device with the sleeve stiffer than the portions of the elongate body without the sleeve. In these variations, the sleeve may include slits and/or openings 134 to ease the transition between the portions of the device with and without the sleeve. The slits or openings may be formed in any suitable shape or pattern, such as, for example, in a spiral shape. In some variations, the slits and/or openings may only be positioned in a transition zone on the sleeve (e.g., on one or both ends of the sleeve), while in other variations the slits and/or openings may be along the entire length of the sleeve. The transition zone may facilitate progressive transition from a less stiff elongate body segment to the more stiff sleeve portion. This may especially provide the benefit of distributing bending forces when the elongate body is flexing.

The sleeve may comprise one or more sleeve openings, which may, in some variations, receive at least a portion of the sensor housing and/or sensor therein and may expose a portion of the sensor housing and thus at least a portion of the sensor positioned therein. A portion of the sensor and/or a portion of the sensor housing may be aligned with the sleeve opening to expose the sensor. For example, a portion of the sensor and/or a portion of the sensor housing may be aligned with the sleeve opening along a depth of the sleeve. For instance, the portion of the sensor and/or the portion of the sensor housing may be positioned underneath the sleeve opening and may be aligned with the opening axially. In some variations, a portion (e.g., a structural component) of the sensor housing may traverse the sleeve opening and may be utilized to secure the sensor housing to the sleeve.

The sensor housing and/or the sensor may be inserted into and/or positioned within the sleeve such that the sensor housing sits within the lumen of the sleeve. Thus, the sleeve and the sensor housing may be configured such that at least a portion of the sensor housing and/or the sensor (e.g., surface of the sensor) may be aligned with an outer surface of the elongate body. Put another way, the sensor housing and/or the sensor may be inset in the sleeve at a depth selected to allow the sensor housing and/or the sensor to be aligned with an outer surface or sidewall of the elongate body. The sensor housing and/or the sensor may be aligned with an outer surface or sidewall of the elongate body such that they are flush. The sleeve opening may allow the sensor to be exposed to the conditions in a patient's body in order to obtain measurements, while the sensor remains protected within the sensor housing. In some variations, the elongate body may include an elongate body opening, window and/or cavity to receive the sensor housing, as described in more detail above.

In some variations, instead of a window, the sleeve may comprise one or more sleeve recessed portions configured to receive at least a portion of the sensor housing therein. When the sleeve is coupled to the elongate body, the sleeve recessed portion may be aligned with the opening and/or window of the elongate body. The sensor housing may be inserted into and/or positioned within the sleeve recessed portion, which may then be received within the opening and/or window of the elongate body. In this way, the sensor housing may be received within the opening and/or window on the elongate body such that it is aligned with an outer surface or sidewall of the elongate body. In some variations, the sensor may not be contained within or encapsulated in a sensor housing. Rather, the sensor may be inserted into and/or positioned within the sleeve such that the sensor sits within the lumen of the sleeve and at least a portion of the sensor may be aligned with an outer surface of the elongate body. In such variations, adhesive may be applied along a perimeter of the sensor to additionally secure the sensor to the sleeve. For example, the sleeve may further comprise one or more holes to provide access point(s) for applying the adhesive to secure the sensor to the sleeve. In some variations, the one or more holes in the sleeve may be used to provide access point(s) for introduction of an adhesive in an amount that wholly or partially encapsulates the sensor in the sensor housing.

### Sensor Housing

The sensor may be contained within a sensor housing configured to protect the sensor from damage. In some variations, the sensor housing may comprise a polymer. In other variations, the sensor housing may comprise a metal. In further variations, the sensor may be encapsulated in the sensor housing. A polymer disposed within the housing may be used to encapsulate (e.g., entirely embed or surround) or at least partially encapsulate (e.g., at least partially embed or surround) the sensor. Entirely or partially encapsulating the sensor may help isolate the sensor from the effects of pressurization, for example, when blood flow control or blood pressure measurement devices including the sensors are used in conjunction with balloons that require pressurization. The polymer may then protect the sensor from pressure within the balloon while also protecting the sensor from materials within the vessel such as blood. Thus, the encapsulating polymer may function, e.g., as a pressure isolator or an insulator, or may have other functions, e.g., as a sealant. Exemplary polymers may include hydrophobic polymers such as RTV silicone (room temperature vulcanizing silicone, Polytetrafluoroethylene (PTFE), polyimide, and Pebax®, and combinations thereof. In one variation, encapsulating the sensor may include forming a layer of polymer, e.g., a layer comprising an RTV silicone or a different polymer, in the sensor housing, placing the sensor on the polymer layer and then curing the polymer. Next, the sensor may be encapsulated by covering the sensor with additional polymer, e.g., RTV silicone, and then curing the polymer. The polymer may have a compliance that allows transmission of pressure signals through it so that physiologic conditions, such as, for example, pressure, may be transmitted through the polymer to the sensor. In some variations, separate seals (e.g., epoxy seals) may be disposed within the sensor housing to protect the sensor from the effects of pressurization.

The sensor may also be encapsulated in the sensor housing to assist with coupling the sensor to the sleeve, and ultimately, to the elongate body. In some variations, the polymer may also serve to maintain the position of the sensor relative to other components (e.g., sensor housing, sleeve, elongate body) and/or an adhesive, e.g., a polymer adhesive, may also be used to perform the same function. In variations in which the sensor is not encapsulated, an adhesive may be used to fix the sensor to the sensor housing.

Additionally or alternatively, the sensor housing may comprise one or more structural components configured to couple the sensor housing to the sleeve. For instance, in some variations, the sensor housing may comprise one or more struts to couple the sensor housing to the sleeve. In these variations, the sleeve opening may be configured to receive the struts and the struts may be attached to the sleeve in the sleeve opening. The struts may be configured to align and/or anchor the sensor within and to the sleeve. The height of the struts may be such that the sensor is appropriately positioned within the lumen of the sleeve and relative to the elongate body to minimize interference with external elements that may affect sensor functionality. For example, the sensor may be exposed just enough to acquire accurate sensor measurement, but may be protected from damage by its placement within the sensor housing and sleeve.

In other variations, the sensor housing may comprise an arm, e.g., a cantilever arm, configured to couple the sensor housing to the sleeve. In these variations, the sensor housing may further comprise a cantilever mount configured to mount the sensor to the cantilever arm. The cantilever arm may be coupled to or otherwise attached to a portion of the sleeve. In some variations, the cantilever arm may comprise the sensor wires.

In some variations, the sensor may be contained within or encapsulated in a sensor housing and the sensor housing may be coupled to the sleeve, such as, by laser welding the sensor housing into the sleeve. The sensor housing may be inserted and/or positioned within the lumen of the sleeve and may be laser welded such that the sensor housing tightly fits within the lumen of the sleeve. The sensor housing may be laser welded into the sleeve along the perimeter of the window and/or opening of the sleeve such that the sensor housing tightly fits within the sleeve.

In variations in which the sleeve comprises a sleeve recessed portion, the sensor housing may be inserted into and/or positioned in the sleeve recessed portion such that the sensor housing may be inset with and/or aligned with an outer surface of the elongate body.

The sensor housing may be formed of any suitable biocompatible material. For example, in some variations, the sensor housing may be formed from a polymer, such as, for example, Polytetrafluoroethylene (PTFE), polyimide, Pebax®, thermoplastic polymers, a combination thereof, and the like. Exemplary thermoplastic polymers may be a polycarbonate, a polycarbonate/acrylonitrile-butadiene-styrene terpolymer blend, etc. In some variations, the sensor housing may be formed from a metal, e.g., stainless steel, a bondable metal alloy, or a combination thereof. In one variation, the sensor housing comprises stainless steel. In variations in which each of the sleeve and the sensor housing comprise a metal (e.g., stainless steel) tube, the tubes may be laser welded together and an opening may be cut through the tubular wall of both the sleeve and the sensor housing to expose or otherwise provide access to the sensor.

In some variations, as discussed above, the sensor may be encapsulated in the sensor housing. Encapsulating the sensor in the housing (e.g., entirely or partially within a polymer) may make the sensor reading less variable to bending forces acting on the sensor and/or may help to isolate the sensor from the effects of pressurization when pressurized devices are employed, e.g., to measure, monitor, and/or adjust blood pressure. Entirely or partially encapsulating the sensor within a polymer may also protect electrical components of the sensor from moisture or fluid intrusion. Thus, encapsulation within a polymer may make the sensor readings more consistent. In some variations, the sensor may be entirely or partially encapsulated within an RTV silicone within a stainless steel housing.

The sensor housing may be variously shaped. For example, the sensor housing may have a tubular, a rectangular, a square, or an ovular shape. In one variation, the sensor housing may be tubular in shape. The dimensions of the sensor housing may be such that the sensor tightly fits in the sensor housing, thereby minimizing the utilization of space on the elongate body. When comprising a tubular shape, the inner diameter of the sensor housing may be between about 0.01 inches and about 0.04 inches. In some variations, the inner diameter of the sensor housing may be for example between about 0.012 inches and about 0.035 inches, between about 0.015 inches and about 0.03 inches, between about 0.017 inches and about 0.025 inches, between about 0.019 inches and about 0.022 inches (including all values and subranges therein). In some variations, the inner diameter of the sensor housing may be about 0.02 inches. In some variations, the outer diameter of the sensor housing may be between about 0.02 inches and about 0.08 inches. In some variations, the outer diameter of the sensor housing may be for example between about 0.021 inches and about 0.06 inches, between about 0.022 inches and about 0.04 inches, between about 0.023 inches and about 0.03 inches, between about 0.024 inches and about 0.027 inches (including all values and subranges therein). In some variations, the outer diameter of the sensor housing may be about 0.025 inches.

### Sensors

As discussed above, the devices described herein may include one or more sensors integrated into an elongate body (e.g., catheter). The sensors may be any sensor useful during a medical procedure, such as, for example pressure sensors, temperature sensors, electrochemical sensors, impedance sensors, and the like. In some variations, the sensors may be solid-state sensors, microelectrochemical system (MEMS) sensors, and/or piezoelectric sensors. In some instances, the sensor may comprise a die or wafer. Any suitable number of sensors (e.g., one, two, three, four, or more) may be integrated into the elongate body to measure physiologic conditions. Some non-limiting examples of physiologic conditions may include blood pressure, heart rate, respiratory rate, intracranial pressure, cerebral oxygenation, cerebral blood flow, electro-encephalographically, and the like. In some embodiments, such as embodiments in which the elongate body is part of a blood flow control device, the one or more sensors may be pressure sensors that measure changes in blood pressure.

In some embodiments, the devices may include two sensors, a first, distal sensor, and a second, proximal sensor, integrated into the elongate body. Each of the distal sensor and the proximal sensor may measure patient physiologic information at a point of intervention to determine the patient's underlying physiology and to provide that information to a user. For example, in a variation in which the distal and proximal sensors may be blood pressure sensors, the distal sensor and the proximal sensor may measure a local blood pressure of the patient at or around the position of the respective sensor. The data from the distal sensor may be used to measure the distal systolic pressure and the distal diastolic pressure of the patient. For instance, distal systolic pressure and distal diastolic pressure may be inferred from a waveform of the blood pressure. Distal systolic pressure may be measured by analyzing peaks of the waveform for a given time duration. Distal diastolic pressure may be measured by analyzing valleys of the waveform for the given time duration. In a similar manner, the data from the proximal sensor may be used to measure the proximal systolic pressure and the proximal diastolic pressure of the patient. For instance, proximal systolic pressure and proximal diastolic pressure may be inferred from a waveform of the blood pressure. Proximal systolic pressure may be measured by analyzing peaks of the waveform for a given time duration. Proximal diastolic pressure may be measured by analyzing valleys of the waveform for the given time duration. In some variations, heart rate, respiratory rate, blood flow rate, cardiac output of the patient, and/or the like may be calculated from the sensor readings obtained from the one or more sensors.

In some variations, medical devices may further include an expandable member coupled to the elongate body. In these variations, sensors may include one or more expandable member sensors that may be integrated into the elongate body. For example, the elongate body may include an expandable member to regulate and/or otherwise control blood flow in a blood vessel of a patient. For instance, the size of the blood vessel may be altered by expanding and/or contracting the expandable member, thereby regulating and/or controlling the blood flow. The expandable member sensor(s) may detect a characteristic of the expandable member, such as, for example, a pressure of fluid and/or compressed gas inside the expandable member. In some variations, expandable member sensor(s) may measure the expandable member pressure. The expandable member pressure may be indicative of the amount of inflation and deflation of the expandable member.

The data from the sensor may be collected continuously or intermittently and may be collected over a defined period of time. The data from the sensor may be collected continuously or intermittently and may be collected over a defined period of time. In some variations, the data from the sensor may be collected continuously, such as for example, every 3 seconds, 4 seconds, 5 seconds, 6 seconds, 7 seconds, 8 seconds, 9 seconds, or 10 seconds (including all values and sub-ranges therein, such as, for example, between about 3 second and about 6 second, about 4 second and about 6 second, or between about 5 second or about 6 second). In some variations, the data from the sensor 118 may be collected every 5 seconds at 200 Hz.

### Controller

In some variations, the devices described herein may further comprise a controller. In some of these variations, the elongate body (e.g., a proximal end of the elongate body) may be coupled to the controller. The controller may be communicatively coupled to the sensors integrated into the elongate body. The controller may comprise a processor. Generally, the processor (e.g., CPU) described here may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor and a storage medium (e.g., memory, flash drive, memory card). The processor may be configured to run and/or execute application processes and/or other modules, processes and/or functions associated with the device.

In some variations, data from the sensors may be analyzed at the controller over a discrete period of time. For instance, the data may be analyzed for example, every 3 seconds, 4 seconds, 5 seconds, 6 seconds, 7 seconds, 8 seconds, 9 seconds, or 10 seconds (including all values and sub-ranges therein, such as, for example, between about 3 second and about 6 second, about 4 second and about 6 second, or between about 5 second or about 6 second).

In some variations, the controller may be communicably coupled to a user interface. For example, the user interface may be a display on the controller. In some variations, the user interface may be a display on any suitable computing device (e.g., computer, smartphone, tablets, etc.) communicably coupled to the controller, via, e.g., the communication device or module described herein.

The user interface may comprise an input device (e.g., touch screen) and output device (e.g., display device) and be configured to receive input data from the sensor. In some variations, an input device may comprise a touch surface for an operator to provide input (e.g., finger contact to the touch surface) corresponding to a control signal. In some variations, a haptic device may be incorporated into one or more of the input and output devices to provide additional sensory output (e.g., force feedback) to the operator.

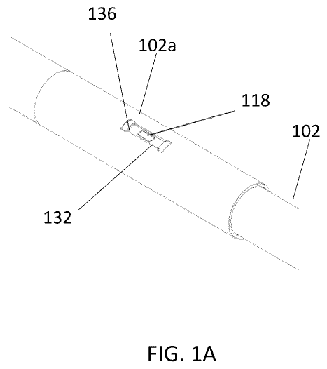

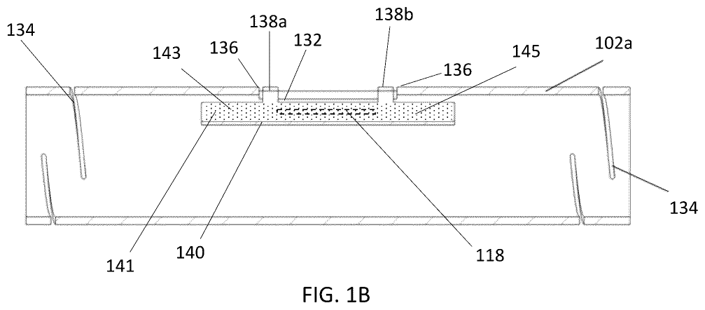

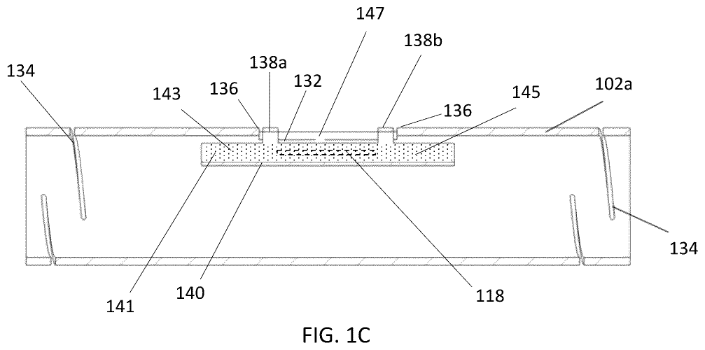

Turning now to the figures, FIG. 1A illustrates an exemplary variation of a sensor 118 integrated into an elongate body 102. The sensor 118 may be positioned in a sensor housing 132 and coupled to or otherwise integrated into the elongate body 102 using a sensor sleeve 102a. The sleeve 102a may include a sleeve opening 136 to receive a portion of the sensor housing 132 therein and to expose a portion of the sensor 118 contained in the sensor housing 132. A portion of the sensor housing 132 may be inserted into and/or positioned within the sleeve opening 136 and the remainder of the sensor housing 132 may sit within the lumen (not shown in FIG. 1A) of the sleeve. In this manner, the sensor housing 132 and/or a sensing surface of the sensor 118 may be aligned with an outer surface or sidewall of the elongate body 102.

FIG. 1B depicts an exemplary variation of a sensor sleeve 102a and a sensor housing 132. The sensor housing 132 may include a body portion 140, which may contain the sensor 118 therein, and one or more struts 138a, 138b extending from the body portion 140. The sensor 118 may be encapsulated (e.g., entirely contained within a polymer) or otherwise sealed within the body portion 140 using a polymer 141 (e.g., an RTV silicone) to make the sensor readings less variable to bending forces acting on the sensor and/or to protect electrical components of the sensor from moisture, fluid intrusion, etc. In some instances, a hydrophobic polymer coating (e.g., a parylene coating) may be applied to protect against moisture and/or fluid intrusion. The physiologic condition the sensor is configured to measure may be transmitted through the polymer to the sensor 118.

Although shown as entirely encapsulated within a polymer 141, in some instances, the polymer 141 may be disposed within the housing 132 in the proximal and/or distal portions, 143 and 145, respectively, outside of struts 138a and 138b. In some variations, an epoxy polymer may be included in the sensor housing portions 143 and 145 to form epoxy seals that may protect the sensor from pressurization.

The sleeve 102a may comprise an opening 136, which may receive at least a portion of the struts 138a, 138b. The struts 138a, 138b may be attached/fastened to the sleeve within the opening 136, thereby coupling the sensor housing 132 to the sleeve 102a. In some variations, such as, for example, variations in which the sensor is encapsulated, the opening 136 may be partially or entirely filled with polymer (e.g., around and/or between the struts 138a, 138b). Fastening the struts to the sensor housing 132 may seal the sensor 118 to the sleeve 102a and the elongate body 102 (not shown).

In some variations, such as that depicted in FIG. 1B, a single opening may be configured to receive multiple struts, while in other variations, the sleeve may comprise a plurality of openings, each of which may receive one or more struts. Moreover, while depicted with two struts, the sensor housing 132 may include any suitable number of struts (e.g., one, two, three, four, or more), to assist in coupling the sensor housing 132 to the sleeve 102a. The length of struts 138a, 138b may be such that the body portion 140 and thus the sensor 118 is positioned at a safe distance from the outer surface of the sleeve and the elongate body. In some variations, the length of the struts 138a, 138b may be between about 0.005 inches and about 0.02 inches, between about 0.0053 inches and about 0.015 inches, between about 0.0055 inches and about 0.01 inches, between about 0.0057 inches and about 0.007 inches, between about 0.0058 inches and about 0.0065 inches (including all values and subranges therein). In some variations, the length of the struts 138a and 138b may be about 0.006 inches. In further variations, as illustrated in FIG. 1C, the sensor housing 132 may include an opening 147 that may provide access to the interior of the housing so that it may be entirely or partially filled with a polymer.

Also depicted in FIG. 1B are slits 134, which may assist with progressively

transitioning the less stiff elongate body segment 102 to the more stiff sleeve 102a portion. As mentioned above, the sensor housing 132 and/or the sensor 118 may be aligned with a sidewall or an outer surface of the elongate body 102, which may optimize the use of space within the elongate body 102 without the sensor housing 132 encroaching on one or more lumens (e.g., an inflation lumen, deflation lumen) of the elongate body 102. More specifically, the sensor housing 132 (e.g., body portion 140), and thus the sensor 118, may be positioned within an opening on the outer surface (e.g., wall) of the elongate body 102. Additionally, sensor wires associated with the sensor 118 may be routed through an annular space (e.g., space between the outer layer and inner layer) of the elongate body (as discussed in FIG. 3), enter one end of the sensor housing 132 and be attached, e.g., by laser welding, to a portion of the sensor 118. Therefore, the sensor 118 and the sensor components (e.g., sensor wires) are positioned so as to optimize the use of space within the elongate body 102.

As discussed above, the sleeve 102a and consequently the sensor housing 132 may add stiffness to the elongate body 102 around the sensor 118, which may assist in protecting the sensor 118 from damage due to exposure to foreign materials within a patient's body. Moreover, the sleeve 102a may include a transition zone, as discussed in more detail above. The sensor housing 132 may be a polymer housing and the sensor 118 may be encapsulated in the polymer housing. Encapsulating the sensor 118 in a polymer housing may make the sensor reading less variable to bending forces acting on the sensor and may make the sensor readings more consistent, thereby improving the accuracy of the sensor 118.

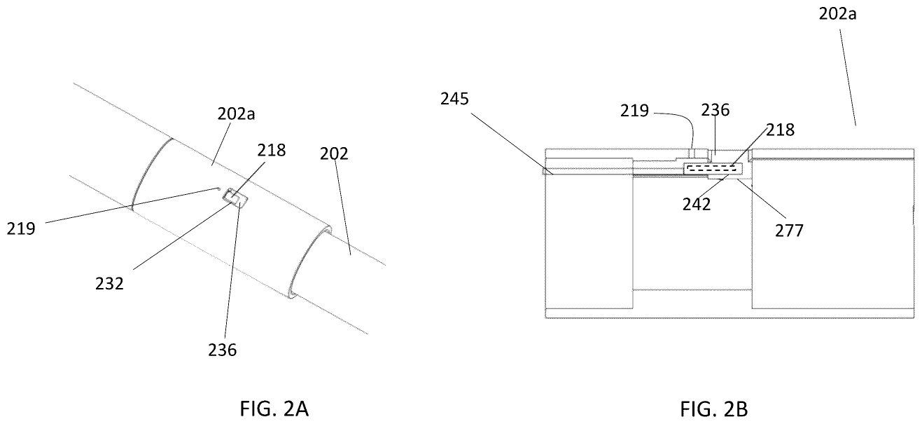

FIGS. 2A illustrates another exemplary variation of a sensor 218 integrated into an elongate body 202. The sensor 218 may be positioned within (e.g., encapsulated within) a sensor housing 232 and coupled to or otherwise integrated into the elongate body 202 using a sensor sleeve 202a. In some variations, the sensor 218 may be directly coupled to, or otherwise integrated into the elongate body 202 using the sensor sleeve 202a. The sensor sleeve 202a may include a window 236 to expose a portion of the sensor housing 232 and/or the sensor 218. The sensor housing 232 and/or the sensor 218 may be positioned within a lumen or cavity in the sleeve 202a and may be aligned with the sleeve opening 236. Put another way, the sensor housing 232 and/or sensor 218 may be inset within the sleeve 202a at a depth selected such that the sensor housing 232 and/or sensor 218 aligns with an outer surface or sidewall of the elongate body 202 and may be positioned such that the sensor 218 is exposed through the window 236. In some variations, an adhesive, e.g., a polymer adhesive, may partially or entirely fill the window 236 in the area surrounding the sensor 218 to entirely encapsulate the sensor 218.

In some variations, the sleeve 202 may include a hole 219. The hole 219 may be an access point for applying an adhesive to additionally secure the sensor 218 to the sleeve 202. The adhesive may be a polymer, e.g., an RTV silicone or another polymer. The adhesive may be applied along the perimeter of the sensor 218 via the hole 219 to additionally secure the sensor 218 to the sleeve. Additionally, the adhesive may be applied proximal of the sensor 218 via the hole 219. For example, the adhesive may be applied along a sensor wire of the sensor 218. The adhesive may also be applied in a manner that partially or entirely fills the window 236 in the area surrounding the sensor 218 to encapsulate the sensor 218 (and/or any die or wafer of the sensor 218.) The physiologic condition the sensor is configured to measure may be transmitted through the adhesive to the sensor 218. In some variations, the sensor 218 may be a piezoelectric sensor.

FIG. 2B depicts a cross-sectional view of the exemplary variation shown in FIG. 2A. For example, FIG. 2B illustrates a cross-sectional view of an exemplary variation of a sensor sleeve 202a. In some variations, the sensor sleeve 202a and/or a sensor housing 232 may comprise a cantilever arm 245 and optionally a cantilever mount 242. The cantilever mount 242 may be configured to couple the sensor 218 to the cantilever arm 245, which may be coupled to the sleeve 202. In this manner, the cantilever arm 245 may support the cantilever mount 242, and thus the sensor 218. While in FIG. 2B the sensor 218 is depicted as being coupled to the cantilever arm 245 via the cantilever mount 242, in some variations, the sensor 218 may be directly coupled to the cantilever arm 245. For example, instead of the cantilever mount 242 coupling the sensor 218 to the cantilever arm 245, the sensor 218 may be directly coupled to the cantilever arm 245.

The positioning of the cantilever arm 245 and/or cantilever mount 242 may be selected to simultaneously expose the sensor 218 to obtain accurate measurements of physiological conditions around the elongate body while protecting the sensor 218 from external elements that may negatively impact sensor functionality. In some variations, the sensor 218 or optionally the cantilever mount 242 may be positioned at a depth of between about 0.02 inches and about 0.06 inches from a top surface of the sleeve 202a. In some variations, the sensor 218 or optionally the cantilever mount 242 may be positioned at a depth of between about 0.021 inches and about 0.05 inches, between about 0.022 inches and about 0.04 inches, between about 0.023 inches and about 0.03 inches, between about 0.024 inches and about 0.027 inches (including all values and subranges therein) from the top surface of the sleeve 202a. In some variations, the sensor 218 or optionally the cantilever mount 242 may be positioned at a depth of about 0.025 inches from the top surface of the sleeve 202a. In some variations, the sensor 218 or optionally the cantilever mount 242 may be positioned such that a clearance gap exists between a bottom surface of the sensor 218 or the bottom surface of the cantilever mount 242 and the sleeve 202a (e.g., the lumen within the sleeve). For example, the sensor 218 or optionally the cantilever mount 242 may be positioned to sit within a lumen of the sleeve 202a such that a clearance gap may exist between a bottom surface of the sensor 218 or bottom surface of the cantilever mount 242 and a segment of the sleeve (e.g., segment 277 in FIG. 2B). In some variations, the clearance gap may be between about 0.005 inches and about 0.02 inches (including all values and subranges therein). In other variations, there may be no clearance gap between the sensor 218 or the bottom surface of the cantilever mount 242 and the sleeve 202a. For example, instead of being open, this area may be filled with an adhesive. In some of these variations, the sensor 218, and in variations comprising a cantilever mount 242, the cantilever mount 242 may be entirely encapsulated within an adhesive.

As discussed above, the sleeve 202a may include a window 236 to expose and/or provide access to the cantilever mount 242 (or a portion thereof) and thus the sensor 218 contained therein or mounted thereon. The sensor 218 or the cantilever mount 242 may be aligned with the window 236. The length and width of the window 236 as well as the depth of the sensor 218 from the top surface of the sleeve 202a may be such that the thrombus formation around the sensor 218 may be minimized while simultaneously protecting the sensor 218 from contact with foreign objects. In some variations, the length of the window 236 may be between about 0.03 inches and about 0.08 inches, between about 0.032 inches and about 0.07 inches, between about 0.034 inches and about 0.06 inches, between about 0.036 inches and about 0.05inches, between about 0.038 inches and about 0.045 inches (including all values and subranges therein). In some variations, the length of the window 236 may be about 0.04 inches. In some variations, the width of the window 236 may be between about 0.015 inches and about 0.04 inches, between about 0.017 inches and about 0.03 inches, between about 0.018 inches and about 0.025 inches (including all values and subranges therein). In some variations, the width of the window 236 may be about 0.02 inches. In some variations, the depth of the window from the top surface of the sleeve 202a may be between about 0.025 inches and about 0.08 inches, between about 0.026 inches and about 0.06 inches, between about 0.027 inches and about 0.06 inches, between about 0.028 inches and about 0.04 inches, between about 0.029 inches and about 0.035 inches (including all values and subranges therein). In some variations, the depth of the window 236 from the top surface of the sleeve 202a may be about 0.03 inches. As mentioned above, in some variations, the window 236 may be partially or entirely filled with an adhesive.

### Exemplary Devices

The exemplary devices described herein may be configured to sense a physiologic condition of a patient (e.g., during a medical procedure). A device may comprise an elongate body comprising one or more sensors integrated into the elongate body, as described in more detail herein. The devices may be configured to identify changes to a patient's physiology, detect when the elongate body transitions from one part of a patient's organ to another part of the organ, provide information about the functioning of a patient's organ, determine when the elongate body is advanced to an incorrect location within the patient's body, etc.

In some variations, the devices may be used to measure (or monitor) blood pressure. The devices may be configured to measure blood pressure at any location within the body. For example, the devices may be used to measure blood pressure in the central arterial or venous vasculature, or the peripheral arterial or venous vasculature. In one variation, the devices may measure blood pressure in the aorta. The pressure measurements may or may not be made using devices that include an expandable member, e.g., an expandable balloon.

In some instances, the device may be a blood flow control device configured to adjust or otherwise control blood flow within a vessel. In these variations, the blood flow control device may comprise an elongate body comprising one or more sensors integrated into the elongate body. The elongate body may further comprise an expandable member (e.g., an expandable balloon). In some variations, the sensors may comprise pressure sensors, and the sensors may be used with the expandable member to maintain a target blood pressure. Fluid such as saline may be pumped to and from a syringe and used to expand and contract the expandable member.

When used with blood flow control devices, the elongate body that is employed may treat shock in patients. The elongate body may be strategically placed within a blood vessel (e.g. aorta) of a patient (e.g., a patient in shock). An expandable member included in the elongate body may be inflated and/or deflated to partially or fully occlude the blood vessel. The amount of occlusion may regulate the blood flow to vital organs in the patient's body. This in turn may help maintain adequate oxygen delivery to the vital organs.

In some variations, an elongate body may be used during procedures for removal of a thrombus from an artery or vein. For example, one or more pressure sensors may be integrated into the elongate body to monitor the pressure in the vein or artery. Some devices with an elongate body and an expandable member (e.g., balloon catheters) may be used to support thrombectomy catheters that are advanced into the vasculature of the brain to remove a blood clot causing a stroke. An expandable member on the end of the elongate body may inflated to occlude a blood vessel based on the sensor readings from the pressure sensor to momentarily stop blood flow while the thrombus or blood clot is removed.

### Exemplary Blood Flow Control Device

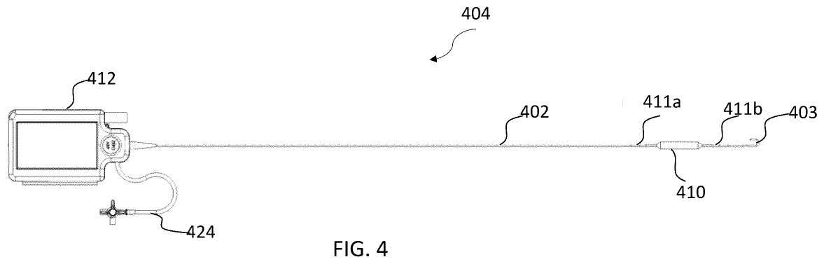

FIG. 4 illustrates an exemplary variation of a blood flow control device 404. The blood flow control device 404 may comprise an elongate body 402, such as any of the elongate bodies 102, 202, and/or 302 described herein, an expandable member 410 coupled to the elongate body 402, and one or more sensors (e.g., 411a and 411b) coupled to or integrated with a shaft of the elongate body 402. The one or more sensors may be any of the sensors described herein with respect to any of the sensors 118 and/or 218, and the one or more sensors may be coupled to or integrated within the shaft of the elongate body using any of the variations described herein. For example, in some variations, the one or more sensors may be contained within a sensor housing and may be coupled to the elongate body via a sleeve, as described in more detail herein, such as with respect to FIGS. 1A-1B and 2A-2B.

### Expandable Member

The expandable member 410 may be one of disposed on, coupled to, integrated with, attached to, and/or affixed to the shaft of the elongate body 402 and a size of the expandable member may be controllable by a controller or a user. For example, the expandable member may be configured to expand and contract and/or inflate and deflate such that the size (e.g., volume) of the expandable member may change during use of the blood flow control device. During use, blood flow may be regulated or otherwise controlled by changing a size of the expandable member 410, thereby altering the area of the blood vessel that is occluded by the expandable member 410. Fluid and/or compressed gas may be delivered through one or more lumens in the elongate body 402 in order to control and/or adjust the size (e.g., volume) of the expandable member 410. Thus, in some variations, the expandable member 410 may be strategically placed within the aorta of a patient and the size of the expandable member 410 may control blood flow through the aorta of the patient such that blood flow distal to expandable member 410 may be impeded to augment blood pressure proximal to expandable member 410. The outer surface of the expandable member 410 may be configured to contact or otherwise interface with the wall(s) of the patient's blood vessel (e.g., at complete occlusion). The expandable member 410 may comprise any suitable elastomeric material (e.g., polyurethane, silicone, etc.). Alternatively, the expandable member may comprise polyester, nylon, etc. In some variations, the expandable member 410 may comprise a shape memory material.

### Sensor(s)

The blood flow control device may comprise two pressure sensors integrated into the elongate body 402. A distal sensor, the position of which is indicated by reference numeral 410b, may be disposed between a tip of the elongate body 402 and the expandable member 410. A proximal sensor, the position of which is indicated by reference numeral 410a, may be disposed between the base of the elongate body 402 (where the elongate body 402 couples to device controller 412) and the expandable member 410. Each of the distal sensor and the proximal sensor may measure patient physiologic condition, such as physiologic information indicative of blood flow through the aorta, to determine the patient's underlying physiology.

In some variations, the distal sensor 410b may be integrated proximal to the expandable 410 member while the proximal sensor 410a may be integrated distal to the expandable member 410. For example, the distal sensor 410b located on the proximal side of the expandable member 410 may be placed at a distance from the expandable member 410 such that the physiologic data collected from the distal sensor 410b may not be disrupted by the blood flow downstream of the expandable member 410. In some variations, the distal sensor 410b may be placed at a distance between about 30 mm and about 10 mm, between about 25 mm and about 15 mm, between about 22 mm and about 18 mm from the expandable member 410. For instance, the distal sensor 410b may be placed approximately 20 mm from the expandable member 410. In some variations, the proximal sensor 410a located on the distal side of the expandable member 410 may be placed between about 30 mm and about 10 mm, between about 25 mm and about 15 mm, or between about 22 mm and about 18 mm from the expandable member. For instance, the proximal sensor 410a may be placed approximately 20 mm from the expandable member 410. As discussed above, in some variations, sensors on the elongate body 402 may be situated at a specific distance from the ends of the expandable member 410 so as to acquire the physiologic data upstream and downstream of the expandable member 410.

Note that the terms “proximal” and “distal,” as used herein in relation to sensor(s) and/or particular localized blood pressure readings, refer to blood flow directionality from the heart. That is, “proximal” is closer to the heart while “distal” is further from the heart. This is not to be confused with the reversed usage of the terms when described from the perspective of a medical device such as a catheter, where the “distal end” of the medical device would commonly be understood as the end with the expandable element 410 furthest from the device controller 412 and the “proximal end” would be understood as the end closer to the operator. In some variations, the blood flow control device may further comprise an expandable member sensor (not shown in FIG. 4) integrated into the elongate body 402. For example, the expandable member sensor may be integrated with the expandable member 410 or with the elongate body 402 within the expandable member 410. In variations in which the expandable member sensor is coupled to the elongate body 402, the sensor may be coupled to or integrated with the elongate body 402 using any of sensor integration configurations and/or structures described herein (e.g., sensor housing, sensor sleeve, etc.) In some variations, the expandable member sensor may be coupled to, integrated with and/or disposed on the device controller 412 and may be fluidly coupled to the expandable member. As discussed above, the expandable member sensor may detect a characteristic of the expandable member.

### Controller

The blood flow control device 404 may comprise and or may be coupled to one or more controllers. For example, the blood flow control device 404 may comprise a device controller 412, which may be coupled to a base of the elongate body 402. The device controller 412 may be communicatively coupled to one or more sensors, such as, for example, the proximal sensor, the distal sensor, and/or the expandable member sensor. For example, the device controller 412 may be electronically coupled to the proximal sensor, the distal sensor and/or the expandable member sensor.

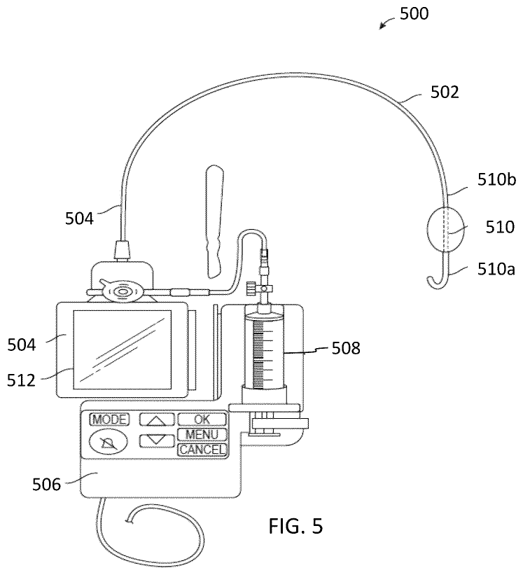

A blood flow control system may comprise a system controller coupled to the blood flow control device (e.g., blood flow control device 404 in FIG. 4). FIG. 5 illustrates an exemplary variation of a blood flow control system 500. In some variations, the blood flow control system may comprise a system controller 506 in addition to the device controller 512 (e.g., device controller 412 in FIG. 4). The system controller 506 may be coupled to the blood flow control device 504, for example, via the device controller 512, or in variations without a device controller 512, via the elongate body 502 directly.

In some variations, the device controller 512 may further comprise a position sensor communicably coupled to a pump 508 (further described below). In some variations, the position sensor may measure a position of a portion of the pump 508. For instance, the position sensor may measure a position of a plunger of a syringe pump 508. The position of the portion of the pump 508 may be used to infer the amount of fluid that has been delivered to and/or removed from the expandable member 510.

Additionally or alternatively, the device controller 512 may comprise a motion sensor (e.g., encoders such as magnetic encoder, optical encoder etc.) communicably coupled to the pump. If the pump 508 is actuated using a motor, the encoder may monitor the movement of the motor, which may be used to determine the amount of inflation and/or deflation in the expandable member 510. In some variations, the motion sensor may be a magnetic encoder. Additionally or alternatively, the motion sensor may be an optical encoder. Additionally or alternatively, at least a portion of the system controller 506 may comprise an optical sensor and/or a contact sensor. The optical sensor and/or contact sensor may be operably coupled to a portion of the pump 508 to determine a position and/or to track the movement of the pump 508. The amount of inflation and/or deflation in the expandable member 510 may be determined based on the position and/or movement of the pump 508. In some variations, the flow sensor described above may determine the amount of inflation and/or deflation in the expandable member 510.

### Pump

As depicted in FIG. 5, the blood flow control system 500 may comprise a pump 508, which may be operably coupled to the expandable member 510 (e.g., expandable member 410 in FIG. 4) to facilitate adjusting a size thereof. The pump 508 may be contained within (e.g., within an open or closed cavity or chamber) or otherwise carried by or coupled to the housing of the device controller 512 or the system controller 506 and may be communicably coupled to one or both of the device controller 512 and the system controller 506. The pump 508 may comprise or otherwise be coupled to an elongate member comprising a lumen (e.g., tubing), which may in turn be coupled to a lumen of the elongate body of the blood flow control device (e.g., an inlet or inflation lumen). In this manner, the pump 508 may be in fluid communication with the expandable member 510.

In some variations, the pump may be fluidly coupled to a valve (e.g., a stopcock valve), which may regulate the flow of fluid and/or compressed gas to the expandable member 510. The size (e.g., volume) of the expandable member may be adjusted using the system and/or device controller 512, 506 and the pump 508.

In summary, the sensors may be configured to measure a physiologic condition of the patient and/or a pressure associated with the expandable member. The controller(s) (e.g., device controller and/or system controller) communicably coupled to the sensors may be configured to receive data from the sensor that may be indicative of the physiologic condition of the patient and/or the pressure associated with the expandable member. The controller(s) may compare the received data with target data and adjust the volume of the expandable member so as to achieve the target data.

### Other Exemplary Devices

### Devices for Navigating Parenchyma of the Brain

In some variations, devices described herein may include one or more sensors integrated into an elongate body configured to navigate in the parenchyma of the brain. For example, a pressure sensor may be integrated into the elongate body as discussed above. The pressure sensor may be housed in a sensor housing. The sensor housing may be coupled to or otherwise attached to the elongate body via a sleeve. The sensor housing may be positioned such that the sensor housing and/or the sensor may be aligned with an outer surface of the elongate body.

The elongate body may be advanced into the parenchyma of the brain. The pressure sensor may measure changes to pressure in the parenchyma as the elongate body navigates in the parenchyma of the brain. Changes to the pressure may indicate that the elongate body has navigated from one part of the brain to another. For instance, the pressure at each part of the brain may be different. Changes to pressure measured by the pressure sensor integrated into the elongate body may indicate the transition of the elongate body from one part of the brain to another. For example, changes to pressure measured by the pressure sensor integrated into the elongate body may indicate the transition of the elongate body from white matter tracts to grey matter, or from the brain parenchyma to the ventricles (e.g., fluid filled areas of the brain), or from brain parenchyma into a blood clot, etc.

### Devices for Monitoring the Gastrointestinal (GI) Tract

In some variations, devices described herein may include one or more sensors integrated into an elongate body that may be used for monitoring the functioning of the gastrointestinal tract (e.g., stomach, large intestine, small intestine). For example, an elongate body comprising one or more integrated sensors, as described in detail herein, may be inserted through the esophagus into the stomach and/or the duodenum. A pressure sensor integrated into the elongate body may measure changes to pressure as the elongate body advances into the stomach. Changes to pressure may provide information about the transition from the esophagus to the stomach across the pylorus and into the small intestine. In some variations, changes to pressure may provide information about the peristalsis of the small bowel.

Devices for Positioning in Endotracheal Tubes

In some variations, a pressure sensor may be integrated into an elongate member that may be positioned in an endotracheal tube, or the elongate body itself may be an endotracheal tube. For example, a pressure sensor may be integrated into the elongate body as discussed in more detail herein. The pressure sensor may provide blood pressure information when a patient is being ventilated. The blood pressure information may provide information about the placement of the endotracheal tube (e.g., esophagus vs. trachea).

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A device configured to monitor a physiologic condition of a patient during a medical procedure, the device comprising:
a sleeve comprising a lumen therethrough and a window;
a sensor housing comprising a sensor therein, wherein the sensor housing is coupled to the sleeve such that the sensor is aligned with the window; and
an elongate body positioned within the lumen of the sleeve,
wherein the sensor is aligned with an outer surface of the elongate body.

2. The device of claim 1, wherein the elongate body comprises an opening and the sensor housing is positioned within the opening of the elongate body.

3. The device of claim 1, wherein the sleeve is configured to add stiffness to the elongate body local to the sensor.

4. The device of claim 1, wherein the sleeve includes a feature configured to distribute bending forces acting on the sensor.

5. The device of claim 4, wherein the sleeve comprises a slit.

6. The device of claim 5, wherein the slit has a spiral geometry.

7. The device of claim 1, wherein the sensor housing comprises a polymer.

8. The device of claim 1, wherein the sensor is encapsulated in the sensor housing.

9. The device of claim 1, wherein the sleeve is configured to seal the sensor to the elongate body.

10. The device of claim 1, wherein the sensor housing comprises a strut coupled to the sleeve.

11. The device of claim 10, wherein the strut is coupled to the sleeve in the window.

12. The device of claim 11, wherein the sensor housing comprises a sealed body portion containing the sensor.

13. The device of claim 12, wherein a length of the strut is between about 0.005 inches and about 0.02 inches.

14. The device of claim 1, wherein the sensor housing is tubular in shape.

15. The device of claim 1, wherein the sensor housing further comprises a cantilever mount to mount the sensor on the sensor housing and a cantilever arm to support the cantilever mount.

16. The device of claim 15, wherein the cantilever mount is aligned with the window.

17. The device of claim 15, wherein the cantilever mount is positioned at a depth of between about 0.25 inches from an outer surface of the sleeve.

18. The device of claim 1, wherein the elongate body comprises an inner layer and an outer layer.

19. The device of claim 18, wherein a sensor wire associated with the sensor is routed between the inner layer and the outer layer.

20. The device of claim 1, wherein the device is a blood flow control device, and the physiologic condition is blood pressure.

21. The device of claim 20, wherein the elongate body further comprises an expandable member coupled to the elongate body.

22. The device of claim 1, wherein the device is configured to be advanced into one or more blood vessels in a brain.

23. The device of claim 22, wherein the device is configured to identify transition of the elongate body from a first part of the brain to a second part of the brain.

24. The device of claim 1, wherein the device is configured to monitor a functioning of a gastrointestinal tract.

25. The device of claim 1, wherein the elongate body is an endotracheal tube.

26. The device of claim 1, wherein the sensor is encapsulated in a polymer within the sensor housing.

27. The device of claim 26, wherein the polymer comprises room temperature vulcanized (RTV) silicone.
